(19) United States
(12) Patent Application Publication
(10) Pub. No.: US 2023/0310813 A1
(43) Pub. Date: Oct. 5, 2023

(54) DIMERIC NAPHTHALIMIDE COATING

(71) Applicant: ALUCENT BIOMEDICAL, INC., Salt Lake City, UT (US)

(72) Inventor: Kevin S. Warner, Salt Lake City, UT (US)

(73) Assignee: ALUCENT BIOMEDICAL, INC., Salt Lake City, UT (US)

(21) Appl. No.: 18/041,989

(22) PCT Filed: Aug. 19, 2021
(86) PCT No.: PCT/US2021/046647

§ 371 Date: Feb. 16, 2023

Related U.S. Application Data
(60) Provisional application No. 63/068,343, filed on Aug. 20, 2020.

(51) Int. Cl. A61M 25/10; A61L 2/00

(52) U.S. Cl. CPC A61M 25/104; A61L 2/0035; A61L 2/007; A61L 2/0094; A61L 2202/24; A61L 2202/21; A61M 2025/1031; A61M 2025/105

(57) ABSTRACT

Stable formulations for a dimeric naphthalimide coating and preparation methods for forming said coating, dimeric naphthalimide coated articles, as well as sterilization and storage-stable packaging thereof.

This application claims priority from U.S. Patent Application No. 63/068,343 filed Aug. 20, 2020, which is hereby incorporated by reference in its entirety.

The present disclosure provides novel stable formulations for a dimeric naphthalimide coating and preparation methods for forming said coating. The present disclosure also relates to dimeric naphthalimide coated articles, as well as sterilization and storage-stable packaging thereof.

Certain dimeric naphthalimide compounds have been previously disclosed. See, e.g., U.S. Pat. No. 6,410,505 B2. For example, a dimeric naphthalimide compound, 2,2′-((ethane-1,2-diylbis(oxy))bis(ethane-2,1-diyl))bis(6-(2-(2-(2-aminoethoxy)ethoxy)ethyl)amino)-1H-benzo[de]isoquinoline-1,3(2H)-dione), also known as 10-8-10 dimer, 6-[2-[2-(2-aminoethoxy)ethoxy]ethylamino]-2-[2-[2-[2-[6-[2-[2-(2-aminoethoxy)ethoxy]ethylamino]-1,3-dioxobenzo[de]isoquinolin-2-yl]ethoxy]ethoxy]ethyl]benzo[de]isoquinoline-1,3-dione; 2,2′-[1,2-ethanediylbix(oxy-2,1-ethanediyl)]bis[6-({2-[2-(2-aminoethoxy)ethoxy]ethyl}amino)-1H-benzo[de]isoquinoline-1,3(2H)-dione]; and 1H-benz[de]isoquinoline-1,3(2H)-dione, 2,2′-[1,2-ethanediylbis(oxy-2,1-ethanediyl)]bis[6-[[2-[2-(2-aminoethoxy)ethoxy]ethyl]amino]-(9C1), and herein referred to as Compound of Formula (I), has been disclosed. Id.

The present disclosure provides novel coating formulations comprising at least one active agent chosen from the Compound of Formula (I):

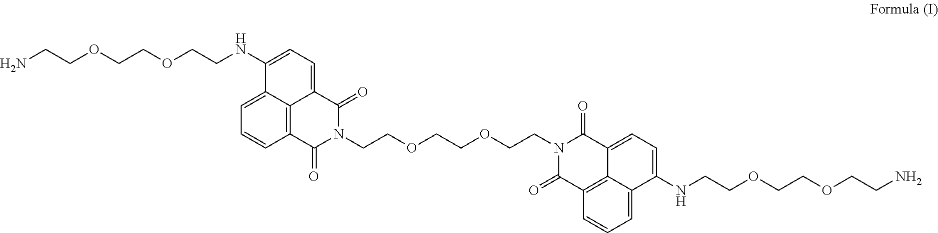 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]

and pharmaceutically acceptable salts thereof.

It was surprisingly and unpredictably discovered that in a solvent system comprising ethanol, water, and acetic acid, a suitable amount of the Compound of Formula (I) and/or pharmaceutically acceptable salts remains solubilized and does not degrade appreciably.

The present disclosure also provides a coated article, wherein the article is coated with a coating comprising at least one active agent chosen from the Compound of Formula (I):

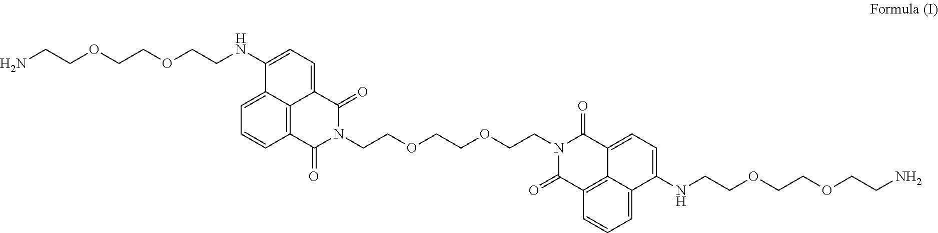 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]

and pharmaceutically acceptable salts thereof. In some embodiments, the coating further comprises at least one antioxidant. In some embodiments, the coating further comprises a Lewis acid. In some embodiments, the coating comprises at least one antioxidant and at least one Lewis acid.

It was discovered that the Compound of Formula (I) and/or pharmaceutically acceptable salts degrades appreciably in a coated article subjected to an ethylene oxide sterilization process. Moreover, it was surprisingly and unpredictably discovered that, when subjected to a radiation sterilization process, there was no change or minimal increase in impurity concentration. In some embodiments, the radiation sterilization process is chosen from nitrogen dioxide sterilization, gamma irradiation, and electron beam.

Further, it was discovered that the Compound of Formula (I) and/or pharmaceutically acceptable salts degrades appreciably in a coated article stored at room temperature. It was surprisingly and unpredictably discovered that, in a coating further comprising at least one antioxidant, the degradation rate over time at room temperature of the Compound of Formula (I) was reduced. It was also surprisingly and unpredictable discovered that, in a coating further comprising at least one Lewis acid, the degradation rate over time at room temperature of the Compound of Formula (I) was reduced. It was also surprisingly and unpredictable discovered that, in a coating further comprising at least one antioxidant and at least one Lewis acid, the degradation rate over time at room temperature of the Compound of Formula (I) was reduced. It was also surprisingly and unpredictable discovered that the increase in impurity concentration was minimal where the coated article was stored in a reduced oxygen and reduced moisture packaging.

### BRIEF DESCRIPTION OF DRAWINGS

FIG. 1 shows HPLC chromatograms of Compound of Formula (I) on a coated balloon subjected to sterilization by ethylene oxide (EtO) treatment, gamma irradiation or electron beam. The chromatograms show minimal increase in the amount of impurities of Compound of Formula (I) for gamma or electron beam sterilization.

FIG. 2 shows the total amount of impurities in a coated article described here.

FIG. 3 depicts a reduction in the formation of impurities in coated articles over time when the coating contains butylated hydroxytoluene (BHT) or tromethamine.

As used herein, the following definitions shall apply unless otherwise indicated.

As used herein, the singular terms “a,” “an,” and “the” include the plural reference unless the context clearly indicates otherwise.

The phrase “and/or,” as used herein, means “either or both” of the elements so conjoined, i.e., elements that are conjunctively present in some cases and disjunctively present in other cases. Thus, as a non-limiting example, “A and/or B”, when used in conjunction with open-ended language such as “comprising” can refer, in some embodiments, to A only (optionally including elements other than B); in other embodiments, to B only (optionally including elements other than A); in yet other embodiments, to both A and B (optionally including other elements); etc.

When an amount is described as “substantially equal to” another amount that means that there is less than 1% difference between the two amounts. For example, where it is described that the total amount of impurities after a certain period of time is substantially equal to the total amount of impurities initially present that means that there was less than 1% increase in the total amount of impurities.

As used herein, “Compound of Formula (I)” includes one or more of the conformational forms of the compound. Unless stated otherwise, compounds depicted herein coexisting with tautomeric forms are within the scope of the disclosure. Additionally, unless stated otherwise, structures depicted herein are also meant to include compounds that differ only in the presence of one or more isotopically enriched atoms. For example, compounds having the depicted structures except for the replacement of hydrogen by deuterium or tritium, or the replacement of a carbon atom by ¹³C- or ¹⁴C-enriched carbon atom are within the scope of this disclosure.

The Compound of Formula (I) may be described by the structure:

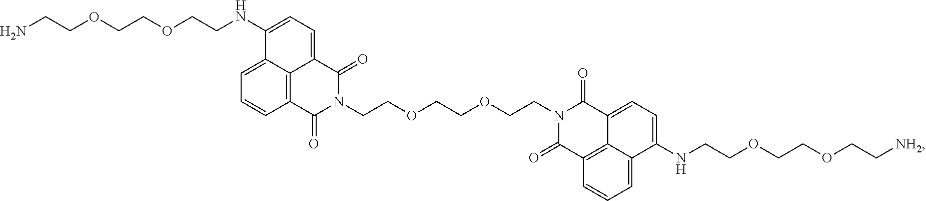 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]

by the chemical names 2,2′-((ethane-1,2-diylbis(oxy))bis(ethane-2,1-diyl))bis(6-((2-(2-(2-aminoethoxy)ethoxy)ethyl)amino)-1H-benzo[de]isoquinoline-1,3(2H)-dione); 6-[2-[2-(2-aminoethoxy)ethoxy]ethylamino]-2-[2-[2-2-[6-[2-[2-(2-aminoethoxy)ethoxy]ethylamino]-1,3-dioxobenzo[de]isoquinolin-2-yl]ethoxy]ethoxy]ethyl]benzo[de]isoquinoline-1,3-dione; 2,2′-[1,2-ethanediylbix(oxy-2,1-ethanediyl)]bis[6-({2-[2-(2-aminoethoxy)ethoxy]ethyl}amino)-1H-benzo[de]isoquinoline-1,3(2H)-dione]; or 1H-benz[de]isoquinoline-1,3(2H)-dione, 2,2′-[1,2-ethanediylbis(oxy-2,1-ethanediyl)]bis[6-[[2-[2-(2-aminoethoxy)ethoxy]ethyl]amino]-(9C1), or by the Chemical Abstract Services (CAS) Registry No. 438200-66-9.

As used herein, the terms “degrade”, “degrades”, and “degradation” may be used interchangeably to refer to the chemical conversion of a chemical entity, e.g., the at least one active agent, into another chemical entity. In some embodiments, degradation of one chemical entity may result in the appearance of or increase in the amount of one or more impurities, which may have undesired effect(s) on a subject who has been administered the one or more impurities.

The degradation of a chemical entity, e.g., the at least one active agent, may be assessed using methods commonly employed in the art to detect and/or distinguish chemical compounds, e.g., liquid chromatography (“LC”) and/or mass spectrometry.

When it is stated herein that a chemical entity, e.g., the at least one active agent, “does not degrade”, that means that there is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 98.75%, at least 99%, at least 99.1%, at least 99.2%, at least 99.25%, at least 99.3%, at least 99.35%, at least 99.4%, at least 99.45%, at least 99.5%, at least 99.55%, at least 99.6%, at least 99.65%, at least 99.7%, at least 99.75%, at least 99.8%, at least 99.85%, at least 99.9%, or at least 99.95% of the initial amount of the chemical entity remaining at the specified time point. In some embodiments, degradation is determined by LC.

As used herein, the term “impurity” refers to an unwanted chemical entity. The genesis of the impurity may be from degradation of a component of a composition described herein, e.g., degradation of at least one active agent, or the impurity may have been introduced during the preparation of the composition. The presence of one or more impurities may be assessed using methods commonly employed in the art to detect and/or distinguish chemical compounds, e.g., liquid chromatography (“LC”) and/or mass spectrometry.

In some embodiments, the total amount of impurities is less than 15%, less than 14%, less than 13%, less than 12%, less than 11%, less than 10%, less than 9%, less than 8%, less than 7%, less than 6%, less than 5%, less than 4%, less than 3%, less than 2.5%, less than 2%, less than 1.5%, less than 1.25%, less than 1%, less than 0.95%, less than 0.9%, less than 0.85%, less than 0.8%, less than 0.75%, less than 0.7%, less than 0.65%, less than 0.6%, less than 0.55%, less than 0.5%, less than 0.45%, less than 0.4%, less than 0.35%, less than 0.3%, less than 0.25%, less than 0.2%, less than 0.15%, less than 0.1%, or less than 0.05% by weight. In some embodiments, the amount of impurities present is determined by LC.

As used herein, the term “LC” means liquid chromatography and includes “HPLC” and “UPLC”, which refer to high performance liquid chromatography and ultra performance liquid chromatography, respectively.

As used herein, the terms “coating composition” or “composition” generally refer to a composition comprising at least one active agent and a solvent system of the present disclosure. The coating composition may be applied to an article, for example, by the methods described in the present disclosure. As used herein, the term “coating” generally refers to the composition after it has been applied to an article to cover at least part of a surface of the article. The coating may be in a liquid state, a solid state, or any intermediate state between a liquid state and a solid state. The solid state (also referred to as “dry state”) may be achieved after the solvent system, or components of the solvent system, have been evaporated or removed partially or fully from the coating composition after the coating composition has been applied to the article.

In some embodiments, the weight percent of the Compound of Formula (I) in a composition or coating can be determined based on LC analysis according to the formula:

$$Percent Assay = \frac{{A}_{S a m p}}{{A}_{S t d}} \times \frac{{C}_{S t d}}{{C}_{calc}} \times 100$$

wherein,
A_{Samp}=Peak area response of Compound of Formula (I) in sample injection
A_{Std}=Mean peak area response of Compound of Formula (I) in all working standard injections
C_{Std}=Concentration of standard, in mg/mL
C_{calc}=Calculated concentration of Compound of Formula (I) in composition or coating, in mg/mL.

In some embodiments, the weight percent of the impurities in a composition or coating can be determined based on LC analysis according to the formula:

$$Percent Impurities ( \% - area ) = \frac{{A}_{Samp I}}{{A}_{I}} \times 100$$

wherein,
A_{Sampl}=Peak area response of impurity in sample injection
Aₗ=Mean peak area response of Compound of Formula (I) in impurity level standard injection.

### Coating Composition

Provided herein is a coating composition comprising at least one active agent and a solvent system. In some embodiments, the coating composition further comprises at least one antioxidant. In some embodiments, the coating composition further comprises at least one Lewis acid. In some embodiments the coating composition further comprises at least one antioxidant and at least one Lewis acid. Provided herein is a method of stabilizing a coating composition comprising dissolving at least one active agent in a solvent system. In some embodiments, the method further comprises dissolving at least one antioxidant in the solvent system. In some embodiments, the method further comprises dissolving at least one Lewis acid in the solvent system. In some embodiments, the method further comprises dissolving at least one antioxidant and at least one Lewis acid in the solvent system. Further provided herein is a method of forming a stabilized coating composition comprising preparing a coating composition comprising at least one active agent and a solvent system. In some embodiments, the coating composition further comprises at least one antioxidant. In some embodiments, the coating composition further comprises at least one Lewis acid. In some embodiments, the coating composition further comprises at least one antioxidant and at least one Lewis acid.

In some embodiments, the at least one active agent is present in an amount ranging from 1% to 10% by weight per volume of the coating composition.

### Coating

Provided herein is a coating comprising at least one active agent. The term “coating” generally refers to the coating composition after it has been applied to at least part of the surface of an article. In some embodiments, for example, soon after being applied to an article, the coating may comprise at least one active agent and a solvent system. In some embodiments, the coating does not comprise a solvent system. In some embodiments, the coating comprises a reduced amount of the solvent system in comparison to the coating composition before application. In some embodiments, the coating comprises only one or more components of the solvent system previously present in the coating composition. In some embodiments, the coating comprises at least one antioxidant. In some embodiments, the coating comprises at least one Lewis acid. In some embodiments, the coating comprises at least one antioxidant and at least one Lewis acid. In some embodiments, the coating comprises at least one active agent, one or more components of a solvent system, and at least one antioxidant. In some embodiments, the coating comprises the compound of formula (I), acetic acid, and tromethamine. In some embodiments, the coating is in a liquid state. In some embodiments, the coating is in a solid state or a dry state. In some embodiments, the coating is in an intermediate state between the liquid state and the solid state or dry state. In some embodiments, the thickness of the coating of a coated article ranges from about 5 μg/mm² and about 10 μg/mm². In some embodiments, the thickness of the coating of a coated article is about 7.5 μg/mm². In some embodiments, the thickness of the coating of a coated article is about 6.5 μg/mm². In some embodiments, the thickness of the coating of a coated article ranges from about 1 μg/mm² and about 5 μg/mm². In some embodiments, the thickness of the coating of a coated article is about 2.3 μg/mm². In some embodiments, the coating comprises about 90-99.8% by weight % of the at least one active agent.

### At Least One Active Agent

Provided herein is a coating composition or coating comprising at least one active agent and a solvent system. In some embodiments, the at least one active agent is chosen from dimeric naphthalimides and pharmaceutically acceptable salts thereof, e.g., the dimeric naphthalimides disclosed in U.S. Pat. No. 6,410,505 B2. In some embodiments, the at least one active agent is a Compound of Formula (I). In some embodiments, the at least one active agent is chosen from a Compound of Formula (I) and pharmaceutically acceptable salts thereof.

In some embodiments, the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium) diacetate. That is the diacetate salt of the free base with the IUPAC name 6-[2-[2-(2-aminoethoxy)ethoxy]ethylamino]-2-[2-[2-[2-[6-[2-[2-(2-aminoethoxy)ethoxy]ethylamino]-1,3-dioxo-benzo[de]isoquinolin-2-yl]ethoxy]ethoxy]ethyl]benzo[de]isoquinoline-1,3-dione.

In some embodiments, the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium).

### A Solvent System

As used herein, the solvent system comprises at least one solvent. In some embodiments, the solvent system comprises ethanol, water, and acetic acid. In some embodiments, the solvent system comprises ethanol in an amount ranging from 80% to 99.2% by volume of the coating composition. In some embodiments, the solvent system comprises water in an amount ranging from 0.6% to 15% by volume of the coating composition. In some embodiments, the solvent system comprises acetic acid in an amount ranging from 0.05 to 5% by volume of the coating composition. In some embodiments, the solvent system comprises acetic acid in an amount of 0.5% by volume of the coating composition. In some embodiments, the solvent system comprises a volume ratio of ethanol to water to acetic acid is 89.5:10:0.5.

In some embodiments, the solvent system comprises ethanol and water. In some embodiments, the solvent system comprises ethanol in an amount ranging from 80% to 97% by volume of the coating composition. In some embodiments, the solvent system comprises water in an amount ranging from 3% to 20% by volume of the coating composition. In some embodiments, the solvent system comprises a volume ratio of ethanol to water is 90:10.

### At Least One Antioxidant

In some embodiments, the coating composition or the coating further comprises at least one antioxidant. Without being bound by any theory, in some embodiments, at least one antioxidant may be added to retard or completely prevent degradation of the at least one active agent, the antioxidant may retard or completely prevent the appearance of impurities in the coating composition, and/or the antioxidant may retard or completely prevent the appearance of impurities in the coating of a coated article.

In some embodiments, the at least one antioxidant is chosen from, butylated hydroxytoluene and tromethamine. In some embodiments, the at least one antioxidant is butylated hydroxytoluene. In some embodiments, the at least one antioxidant is tromethamine.

In some embodiments, the at least one antioxidant is present in an amount ranging from 0.05-1% by weight per volume of the coating composition.

### At Least One Lewis Acid

In some embodiments, the coating composition or coating further comprises at least one Lewis acid. Without being bound by any theory, in some embodiments, at least one Lewis acid may be added to retard or completely prevent degradation of the at least one active agent, the Lewis acid may retard or completely prevent the appearance of impurities in the coating composition, and/or the Lewis acid may retard or completely prevent the appearance of impurities in the coating of a coated article.

In some embodiments, the at least one Lewis acid is Na+. In some embodiments, the at least one Lewis acid is in the form of NaCl. In some embodiments, the at least one Lewis acid is in the form of KCl. In some embodiments, the at least one Lewis acid is K⁺, Mg²⁺, or Ca²⁺.

In some embodiments, the coating composition or coating further comprises at least one antioxidant and at least one Lewis acid.

### Coating Composition and Coating Properties

### Solubility of Active Agent

In some embodiments, provided herein is a coating composition or a coating comprising at least one active agent, wherein at least 2% by weight per weight (w/w) remains solubilized in the coating composition or the coating for at least 8 hours after preparation of the coating composition. In some embodiments, provided herein is a coating composition or coating comprising at least one active agent, wherein at least 2% w/w remains solubilized in the coating composition or the coating for 10 hours, 12 hours, 16 hours, 18 hours, 20 hours, 24 hours, 30 hours, 36 hours or 48 hours after preparation of the coating composition. In some embodiments, an amount of about 2%, 2.2%, 2.4%, 2.6%, 2.8%, 3%, 3.2%, 3.4%, 3.6%, 3.8% or 4% w/w of the at least one active agent remains solubilized in the coating composition or the coating for at least 8 hours after preparation of the coating composition. In some embodiments, the amount solubilized is determined by LC.

A coating composition or coating where a certain amount, e.g. at least 2% w/w, of the at least one active agent remains solubilized after preparation of the coating composition may be advantageous, for example, because the amount solubilized in the coating composition or the coating may affect the quantity of the at least one active agent in the final coated article.

In some embodiments, provided herein is a coating composition or coating comprising at least one active agent, wherein at least 2% w/w remains solubilized in the coating composition or the coating for at least 8 hours after preparation of the coating composition. In some embodiments, at least 2% w/w remains solubilized in the coating composition or the coating for 10 hours after preparation of the coating composition. In some embodiments, at least 2% w/w remains solubilized in the coating composition or the coating for 12 hours after preparation of the coating composition. In some embodiments, the at least 2% w/w remains solubilized in the coating composition or the coating for 14 hours after preparation of the coating composition. In some embodiments, at least 2% w/w remains solubilized in the coating composition or the coating for 16 hours after preparation of the coating composition. In some embodiments, at least 2% w/w remains solubilized in the coating composition or the coating for 18 hours after preparation of the coating composition. In some embodiments, at least 2% w/w remains solubilized in the coating composition or the coating for 20 hours after preparation of the coating composition. In some embodiments, the amount solubilized is determined by LC.

In some embodiments, at least 2% w/w remains solubilized in the coating composition or the coating for 1 day (24 hours) after preparation of the coating composition. In some embodiments, at least 2% w/w remains solubilized in the coating composition or the coating for 30 hours after preparation of the coating composition. In some embodiments, at least 2% w/w remains solubilized in the coating composition or the coating for 36 hours after preparation of the coating composition. In some embodiments, at least 2% w/w remains solubilized in the coating composition or the coating for 2 days (48 hours) after preparation of the coating composition. In some embodiments, the amount solubilized is determined by LC.

In some embodiments, provided herein is a coating composition or coating comprising at least one active agent, wherein at least 2% w/w remains solubilized in the coating composition or the coating for at least 8 hours after preparation of the coating composition. In some embodiments, an amount of about 2.2% w/w of the at least one active agent remains solubilized in the composition or the coating for at least 8 hours after preparation of the coating composition. In some embodiments, an amount of about 2.4% w/w of the at least one active agent remains solubilized in the composition or the coating for at least 8 hours after preparation of the coating composition. In some embodiments, an amount of about 2.6% w/w of the at least one active agent remains solubilized in the composition or the coating for at least 8 hours after preparation of the coating composition. In some embodiments, an amount of about 2.8% w/w of the at least one active agent remains solubilized in the composition or the coating for at least 8 hours after preparation of the coating composition.

In some embodiments, provided herein is a coating composition or coating comprising at least one active agent, wherein at least 3% w/w remains solubilized in the coating composition or the coating for at least 8 hours after preparation of the coating composition. In some embodiments, an amount of about 3% w/w of the at least one active agent remains solubilized in the composition or the coating for at least 8 hours after preparation of the coating composition. In some embodiments, an amount of about 3.2% w/w of the at least one active agent remains solubilized in the composition or the coating for at least 8 hours after preparation of the coating composition. In some embodiments, an amount of about 3.4% w/w of the at least one active agent remains solubilized in the composition or the coating for at least 8 hours. In some embodiments, an amount of about 3.6% w/w of the at least one active agent remains solubilized in the composition or the coating for at least 8 hours after preparation of the coating composition. In some embodiments, an amount of about 3.8% w/w of the at least one active agent remains solubilized in the composition or the coating for at least 8 hours after preparation of the coating composition.

In some embodiments, provided herein is a coating composition or coating comprising at least one active agent, wherein at least 4% w/w remains solubilized in the coating composition or the coating for at least 8 hours after preparation of the coating composition. In some embodiments, an amount of about 4% w/w of the at least one active agent remains solubilized in the composition or the coating for at least 8 hours after preparation of the coating composition.

In some embodiments, provided herein is a coating composition or a coating comprising at least one active agent, wherein at least 5% or about 5% by weight per weight (w/w) of the at least one active agent remains solubilized in the coating composition or the coating for at least 8 hours after preparation of the coating composition. In some embodiments, provided herein is a coating composition or a coating comprising at least one active agent, wherein at least 5% w/w or about 5% w/w of the at least one active agent remains solubilized in the coating composition or the coating for 10 hours, 12 hours, 16 hours, 18 hours, 20 hours, 24 hours, 30 hours, 36 hours or 48 hours after preparation of the coating composition.

A coating composition or a coating where a certain amount, e.g. at least 5% w/w or about 5% w/w, of the at least one active agent remains solubilized after preparation of the coating composition may be advantageous, for example, because the amount solubilized in the coating composition or the coating may affect the quantity of the at least one active agent in the final coated article.

In some embodiments, provided herein is a coating composition or an active agent comprising at least one active agent, wherein at least 5% w/w or about 5% w/w of the at least one active agent remains solubilized in the coating composition or the coating for at least 8 hours after preparation of the coating composition. In some embodiments, at least 5% w/w or about 5% w/w remains solubilized in the coating composition or the coating for 10 hours after preparation of the coating composition. In some embodiments, at least 5% w/w or about 5% w/w remains solubilized in the coating composition or the coating for 12 hours after preparation of the coating composition. In some embodiments, at least 5% w/w or about 5% w/w remains solubilized in the coating composition or the coating for 14 hours after preparation of the coating composition. In some embodiments, at least 5% w/w or about 5% w/w remains solubilized in the coating composition or the coating for 16 hours after preparation of the coating composition. In some embodiments, at least 5% w/w or about 5% w/w remains solubilized in the coating composition or the coating for 18 hours after preparation of the coating composition. In some embodiments, at least 5% w/w or about 5% w/w remains solubilized in the coating composition or the coating for 20 hours after preparation of the coating composition. In some embodiments, the amount solubilized is determined by LC.

In some embodiments, at least 5% w/w or about 5% w/w remains solubilized in the coating composition or the coating for 1 day (24 hours) after preparation of the coating composition. In some embodiments, at least 5% w/w or about 5% w/w remains solubilized in the coating composition or the coating for 30 hours after preparation of the coating composition. In some embodiments, at least 5% w/w or about 5% w/w remains solubilized in the coating composition or the coating for 36 hours after preparation of the coating composition. In some embodiments, at least 5% w/w or about 5% w/w remains solubilized in the coating composition or the coating for 2 days (48 hours) after preparation of the coating composition. In some embodiments, the amount solubilized is determined by LC.

In some embodiments, provided herein is a coating composition or a coating comprising at least one active agent, wherein up to about 10% w/w of the at least one active agent remains solubilized in the coating composition or the coating for at least 8 hours after preparation of the coating composition. In some embodiments, provided herein is a coating composition or a coating comprising at least one active agent, wherein up to about 10% w/w of the at least one active agent remains solubilized in the coating composition or the coating for 10 hours, 12 hours, 16 hours, 18 hours, 20 hours, 24 hours, 30 hours, 36 hours or 48 hours after preparation of the coating composition.

A coating composition or a coating where a certain amount, e.g. up to about 10% w/w, of the at least one active agent remains solubilized after preparation of the coating composition may be advantageous, for example, because the amount solubilized in the coating composition or the coating may affect the quantity of the at least one active agent in the final coated article.

In some embodiments, provided herein is a coating composition or a coating comprising at least one active agent, wherein up to about 10% w/w of the at least one active agent remains solubilized in the coating composition or the coating for at least 8 hours after preparation of the coating composition. In some embodiments, up to about 10% w/w remains solubilized in the coating composition or the coating for 10 hours after preparation of the coating composition. In some embodiments, up to about 10% w/w remains solubilized in the coating composition or the coating for 12 hours after preparation of the coating composition. In some embodiments, up to about 10% w/w remains solubilized in the coating composition or the coating for 14 hours after preparation of the coating composition. In some embodiments, up to about 10% w/w remains solubilized in the coating composition or the coating for 16 hours after preparation of the coating composition. In some embodiments, up to about 10% w/w remains solubilized in the coating composition or the coating for 18 hours after preparation of the coating composition. In some embodiments, up to about 10% w/w remains solubilized in the coating composition or the coating for 20 hours after preparation of the coating composition. In some embodiments, the amount solubilized is determined by LC.

In some embodiments, up to about 10% w/w remains solubilized in the coating composition or the coating for 1 day (24 hours) after preparation of the coating composition. In some embodiments, up to about 10% w/w remains solubilized in the coating composition or the coating for 30 hours after preparation of the coating composition. In some embodiments, up to about 10% w/w remains solubilized in the coating composition or the coating for 36 hours after preparation of the coating composition. In some embodiments, up to about 10% w/w remains solubilized in the coating composition or the coating for 2 days (48 hours) after preparation of the coating composition. In some embodiments, the amount solubilized is determined by LC.

### Coating Composition and Coating Stability

In some embodiments, provided herein is a coating composition or a coating comprising at least one active agent, wherein the at least one active agent does not degrade for 10 hours, 12 hours, 16 hours, 18 hours, 20 hours, 24 hours, 30 hours, 36 hours or 48 hours after preparation of the coating composition. In some embodiments, degradation is determined by LC.

A coating composition or coating where the at least one active agent does not degrade for a specific period of time after preparation of the coating composition may be advantageous, for example, because (1) the concentration of the at least one active agent is predictable; (2) degradation products of the at least one active agent may have deleterious effects if a coated article comprising the coating is administered to a subject; and/or (3) the coating composition may be prepared in advance of manufacturing of a coated article and excess coating composition may be stored for later use.

In some embodiments, the at least one active agent does not degrade 10 hours after preparation of the coating composition. In some embodiments, the at least one active agent does not degrade 12 hours after preparation of the coating composition. In some embodiments, the at least one active agent does not degrade 14 hours after preparation of the coating composition. In some embodiments, the at least one active agent does not degrade 16 hours after preparation of the coating composition. In some embodiments, the at least one active agent does not degrade 18 hours after preparation of the coating composition. In some embodiments, the at least one active agent does not degrade 20 hours after preparation of the coating composition. In some embodiments, degradation is determined by LC.

In some embodiments, the at least one active agent does not degrade for 1 day (24 hours) after preparation of the coating composition. In some embodiments, the at least one active agent does not degrade 30 hours after preparation of the coating composition. In some embodiments, the at least one active agent does not degrade 36 hours after preparation of the coating composition. In some embodiments, the at least one active agent does not degrade for 2 days (48 hours) after preparation of the coating composition. In some embodiments, degradation is determined by LC.

In some embodiments, there is 0% to less than 15%, less than 14%, less than 13%, less than 12%, less than 11° A, less than 10%, less than 9%, less than 8%, less than 7%, less than 6%, less than 5%, less than 4.5%, less than 4%, less than 3.5%, less than 3%, less than 2.5%, less than 2%, less than 1.5%, less than 1%, less than 0.75%, less than 0.5%, less than 0.45%, less than 0.4%, less than 0.35%, less than 0.3%, less than 0.25%, less than 0.2%, less than 0.15%, less than 0.1%, or less than 0.05% increase in the total amount of impurities present in the coating composition or the coating 10 hours, 12 hours, 16 hours, 18 hours, 20 hours, 24 hours, 30 hours, 36 hours, 2 days, 3 days, 4 days, 5 days, 6 days, 1 week, 2 weeks, 3 weeks, 5 weeks, 10 weeks, 20 weeks, or 1 year after preparation of the coating composition. In some embodiments, the amount of impurities is determined by LC.

In some embodiments, there is 0% to less than 5% increase in the total amount of impurities present in the coating composition or the coating 24 hours after preparation of the coating composition. In some embodiments, there is 0% to less than 1% increase in the total amount of impurities present in the coating composition or the coating 24 hours after preparation of the coating composition. In some embodiments, there is 0% to less than 0.5% increase in the total amount of impurities present in the coating composition or the coating 24 hours after preparation of the coating composition. In some embodiments, there is 0% to less than 0.25% increase in the total amount of impurities present in the coating composition or the coating 24 hours after preparation of the coating composition. In some embodiments, there is 0% to less than 0.05% increase in the total amount of impurities present in the coating composition or the coating 24 hours after preparation of the coating composition. In some embodiments, the amount of impurities is determined by LC.

In some embodiments, there is 0% to less than 5% increase in the total amount of impurities present in the coating composition or the coating 2 days after preparation of the coating composition. In some embodiments, there is 0% to less than 2.5% increase in the total amount of impurities present in the coating composition or the coating 2 days after preparation of the coating composition. In some embodiments, there is 0% to less than 1% increase in the total amount of impurities present in the coating composition or the coating 2 days after preparation of the coating composition. In some embodiments, there is 0% to less than 0.5% increase in the total amount of impurities present in the coating composition or the coating 2 days after preparation of the coating composition. In some embodiments, there is 0% to less than 0.25% increase in the total amount of impurities present in the coating composition or the coating 2 days after preparation of the coating composition. In some embodiments, there is 0% to less than 0.05% increase in the total amount of impurities present in the coating composition or the coating 2 day after preparation of the coating composition. In some embodiments, the amount of impurities is determined by LC.

In some embodiments, provided herein is a coating composition or a coating, wherein the total amount of impurities in the coating composition or the coating 10 hours, 12 hours, 16 hours, 18 hours, 20 hours, 24 hours, 30 hours, 36 hours, 2 days, 3 days, 4 days, 5 days, 6 days, or 1 week after preparation of the coating composition is less than five-fold the total amount of impurities initially present in the coating composition or the coating. In some embodiments, the amount of impurities is determined by LC.

In some embodiments, provided herein is a coating composition or a coating, wherein the total amount of impurities in the coating composition or the coating 10 hours, 12 hours, 16 hours, 18 hours, 20 hours, 24 hours, 30 hours, 36 hours, 2 days, 3 days, 4 days, 5 days, 6 days, or 1 week after preparation of the coating composition is less than four-fold the total amount of impurities initially present in the coating composition or the coating. In some embodiments, the amount of impurities is determined by LC.

In some embodiments, provided herein is a coating composition or a coating, wherein the total amount of impurities in the coating composition or the coating 10 hours, 12 hours, 16 hours, 18 hours, 20 hours, 24 hours, 30 hours, 36 hours or 48 hours after preparation of the coating composition is less than three-fold the total amount of impurities initially present in the coating composition or the coating. In some embodiments, the amount of impurities is determined by LC.

In some embodiments, provided herein is a coating composition or a coating, wherein the total amount of impurities in the coating composition 10 hours, 12 hours, 16 hours, 18 hours, 20 hours, 24 hours, 30 hours, 36 hours, 2 days, 3 days, 4 days, 5 days, 6 days, or 1 week after preparation of the coating composition is less than two-fold the total amount of impurities initially present in the coating composition or the coating. In some embodiments, the amount of impurities is determined by LC.

In some embodiments, provided herein is a coating composition or a coating, wherein the total amount of impurities in the coating composition or the coating 10 hours, 12 hours, 16 hours, 18 hours, 20 hours, 24 hours, 30 hours, 36 hours, 2 days, 3 days, 4 days, 5 days, 6 days, or 1 week after preparation of the coating composition is substantially equal to the total amount of impurities initially present in the coating composition or the coating. In some embodiments, the amount of impurities is determined by LC.

### Coated Article

Provided herein is a method of preparing an article coated with the coating composition comprising preparing the coating composition comprising at least one active agent and a solvent system, applying the coating composition to at least a portion of a surface of an article, and evaporating the solvent system to form a coated article comprising a coating.

In some embodiments, the article is a medical device. In some embodiments the article is a balloon, for example an angioplasty balloon. In some embodiments, a coated balloon is prepared by coating a balloon with the coating composition, thereby forming a coating. In some embodiments, an angioplasty balloon is prepared by coating an angioplasty balloon with the coating composition, thereby forming a coating.

### Sterilization Method

Provided herein is a method of sterilizing a coated article, wherein the coating of the coated article comprises at least one active agent. Further provided herein is a sterilized coated article produced by subjecting a coated article to the method of sterilizing. In some embodiments, the method of sterilizing the coated article comprises subjecting the coated article to a radiation sterilization process. In some embodiments the radiation sterilization process is chosen from nitrogen dioxide sterilization, gamma irradiation and electron beam processing.

In some embodiments, the at least one active agent is chosen from dimeric naphthalimides and pharmaceutically acceptable salts thereof, e.g., the dimeric naphthalimides disclosed in U.S. Pat. No. 6,410,505 B2. In some embodiments, the at least one active agent is a Compound of Formula (I). In some embodiments, the at least one active agent is chosen from a Compound of Formula (I) and pharmaceutically acceptable salts thereof.

In some embodiments, the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium) diacetate. In some embodiments, the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium).

In some embodiments, provided herein is a method of sterilizing a coated article, wherein there is 0% to less than 30%, less than 29%, less than 25%, less than 24%, less than 23%, less than 22%, less than 21%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4.5%, less than 4%, less than 3.5%, less than 3%, less than 2.5%, less than 2%, less than 1.5%, less than 1%, less than 0.75%, less than 0.5%, less than 0.45%, less than 0.4%, less than 0.35%, less than 0.3%, less than 0.25%, less than 0.2%, less than 0.15%, less than 0.1%, or less than 0.05% increase in the total amount of impurities present in the coating after sterilization. In some embodiments, the total amount of impurities is measured 1 hour, 2 hours, 6 hours, 12 hours, 24 hours, 5 days, 1 week, 2 weeks, 3 weeks, 4 weeks, 2 months, 3 months, 4 months, 5 months, 6 months, 7 months, 8 months, 9 months, 10 months, 11 months, 12 months, 16 months, 18 months, 20 months, 24 months, or 36 months after sterilization of the coated article.

In some embodiments, degradation is determined by LC.

Without being bound by any theory, in some embodiments, a method of sterilizing a coated article where the at least one active agent does not degrade may be advantageous, for example, the concentration of the at least one active agent is predictable; and/or degradation products of the at least one active agent may have deleterious effects if a coated article comprising the coating is administered to a subject.

In some embodiments, there is 5% to less than 15% increase in the total amount of impurities present in the coating after sterilization. In some embodiments, there is less than 15% increase in the total amount of impurities present in the coating after sterilization. In some embodiments, there is less than 10% increase in the total amount of impurities present in the coating after sterilization. In some embodiments, there is less than 5% increase in the total amount of impurities present in the coating after sterilization.

In some embodiments, there is 0% to less than 5% increase in the total amount of impurities present in the coating after sterilization. In some embodiments, there is 0% to less than 2.5% increase in the total amount of impurities present in the coating after sterilization. In some embodiments, there is 0% to less than 0.5% increase in the total amount of impurities present in the coating after sterilization. In some embodiments, there is 0% to less than 1% increase in the total amount of impurities present in the coating after sterilization. In some embodiments, there is 0% to less than 0.5% increase in the total amount of impurities present in the coating after sterilization. In some embodiments, there is 0% to less than 0.25% increase in the total amount of impurities present in the coating after sterilization. In some embodiments, there is 0% to less than 0.05% increase in the total amount of impurities present in the coating after sterilization. In some embodiments, the amount of impurities is determined by LC

### Packaging

Provided herein is a method for preparing an article coated with a stabilized coating composition comprising at least one active agent. In some embodiments, the method comprises preparing a coating composition comprising at least one active agent and a solvent system, applying the coating composition to at least a portion of a surface of an article, evaporating the solvent system to form a coated article comprising a coating, and packaging the coated article in a sealable container. In some embodiments, the method further comprises the steps of reducing the concentration of oxygen from within the container, adding at least one desiccant to the container, and sealing the oxygen-purged container comprising the coated article and at least one desiccant.

Provided herein is a method for stabilizing the coating of an article having a coating comprising at least one active agent. In some embodiments, the method comprises preparing a coating composition comprising at least one active agent and a solvent system, coating at least a portion of a surface of an article with the coating composition to form a coated article comprising a coating, and packaging the coated article in a sealable container. In some embodiments, the method further comprises the steps of reducing the concentration of oxygen from within the container, adding at least one desiccant to the container, and sealing the oxygen-purged container comprising the coated article and at least one desiccant.

Provided herein is a storage-stable packaged article coated with a coating comprising at least one active agent. In some embodiments, the packaged article comprises a sealable container containing, in a reduced oxygen atmosphere, the article coated with a coating comprising at least one active agent, and at least one desiccant.

In some embodiments, the at least one active agent is chosen from dimeric naphthalimides and pharmaceutically acceptable salts thereof, e.g., the dimeric naphthalimides disclosed in U.S. Pat. No. 6,410,505 B2. In some embodiments, the at least one active agent is a Compound of Formula (I). In some embodiments, the at least one active agent is chosen from a Compound of Formula (I) and pharmaceutically acceptable salts thereof.

In some embodiments, the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium) diacetate. In some embodiments, the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium).

In some embodiments, the coating of the coated article comprises about 90-99.8% by weight % of the at least one active agent.

In some embodiments, the coating of the coated article further comprises at least one antioxidant. In some embodiments, the at least one antioxidant is chosen from butylated hydroxytoluene and tromethamine.

In some embodiments, the coating of the coating article further comprises at least one Lewis acid. In some embodiments, the at least one Lewis acid is Na⁺. In some embodiments, the at least one Lewis acid is in the form of NaCl. In some embodiments, the at least one Lewis acid is in the form of KCl. In some embodiments, the at least one Lewis acid is K⁺, Mg²⁺, or Ca²⁺.

In some embodiments the coating of the coated article comprises at least one antioxidant and at least one Lewis acid.

In some embodiments, the method comprises the step of reducing the concentration of oxygen from within the container. In some embodiments, the concentration of oxygen within the container is reduced by purging with nitrogen.

In some embodiments, the method comprises the step of adding at least one desiccant to the container. In some embodiments, the desiccant is a silica gel packet.

In some embodiments, the method comprises the step of packaging the coated article in a sealable containing. In some embodiments, the sealable container is a foil pouch.

In some embodiments, provided herein is a packaged article, wherein there is 0% to less than 30%, less than 29%, less than 25%, less than 24%, less than 23%, less than 22%, less than 21%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4.5%, less than 4%, less than 3.5%, less than 3%, less than 2.5%, less than 2%, less than 1.5%, less than 1%, less than 0.75%, less than 0.5%, less than 0.45%, less than 0.4%, less than 0.35%, less than 0.3%, less than 0.25%, less than 0.2%, less than 0.15%, less than 0.1%, or less than 0.05% increase in the total amount of impurities present in the coating 24 months after packaging. In some embodiments, degradation is determined by LC.

In some embodiments, provided herein is a packaged article, wherein there is 0% to less than 30%, less than 29%, less than 25%, less than 24%, less than 23%, less than 22%, less than 21%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4.5%, less than 4%, less than 3.5%, less than 3%, less than 2.5%, less than 2%, less than 1.5%, less than 1%, less than 0.75%, less than 0.5%, less than 0.45%, less than 0.4%, less than 0.35%, less than 0.3%, less than 0.25%, less than 0.2%, less than 0.15%, less than 0.1%, or less than 0.05% increase in the total amount of impurities present in the coating 36 months after packaging. In some embodiments, degradation is determined by LC.

Without being bound by any theory, in some embodiments, the packaged article where the at least one active agent does not degrade may be advantageous, for example, the concentration of the at least one active agent is predictable; and/or degradation products of the at least one active agent may have deleterious effects if a coated article comprising the coating is administered to a subject.

In some embodiments, there is 5% to less than 15% increase in the total amount of impurities present in the coating after packaging. In some embodiments, there is less than 15% increase in the total amount of impurities present in the coating after packaging. In some embodiments, there is less than 10% increase in the total amount of impurities present in the coating after packaging. In some embodiments, there is less than 5% increase in the total amount of impurities present in the coating after packaging.

In some embodiments, there is 0% to less than 5% increase in the total amount of impurities present in the coating after packaging. In some embodiments, there is 0% to less than 2.5% increase in the total amount of impurities present in the coating after packaging. In some embodiments, there is 0% to less than 0.5% increase in the total amount of impurities present in the coating after packaging. In some embodiments, there is 0% to less than 1% increase in the total amount of impurities present in the coating after packaging. In some embodiments, there is 0% to less than 0.5% increase in the total amount of impurities present in the coating after packaging. In some embodiments, there is 0% to less than 0.25% increase in the total amount of impurities present in the coating after packaging. In some embodiments, there is 0% to less than 0.05% increase in the total amount of impurities present in the coating after packaging. In some embodiments, the amount of impurities is determined by LC

In order that the disclosure herein may be more fully understood, the following examples are set forth. It should be understood that these examples are for illustrative purposes only and are not to be construed as limiting this disclosure in any manner.

### EXAMPLES

### Abbreviations

The following abbreviations are used herein:

HPLC=high pressure liquid chromatography

TFA=trifluoroacetic acid

UPLC=ultra performance liquid chromatography

General methods and experimental details for preparing the coating composition of the present disclosure are set forth below.

### Reagents

The excipients and reagents used in the following examples are compendial or pharmaceutical grade, or higher.

### Purity Assessment

The purity of a composition was analyzed by UPLC or HPLC.

### Example 1: Coating Composition

Table 1 lists exemplary and comparative solvent systems.

TABLE 1
Solvent Systems

| | 89.5% Ethanol/ | 99.5% | | | | | | |
|---|---|---|---|---|---|---|---|---|
| | 10% water/ | Ethanol/ | 99% | Ethanol/ | | | | |
| | 0.5% acetic | 0.5% acetic | Ethanol/ | Acetone/ | Ethanol/ | Ethanol/ | Ethanol/ | Ethanol/ |
| Solvent Criteria | acid | acid | 1% Acetone | BHT | MEK | IPA | EtAc | THF |
| 10-8-10 stability over | ✓ | ✓ | X | X | X | ✓ | | |
| 8 hours | | | | | | | | |
| Solubility >2% w/w | ✓ | X | ✓ | ✓ | ✓ | X | X | X |
| Compatible with | ✓ | ✓ | ✓ | ✓ | ✓ | ✓ | ✓ | ✓ |
| catheter material | | | | | | | | |
| Listed in ICH Q3C | ✓ | ✓ | ✓ | ✓ | ✓ | ✓ | ✓ | ✓ |
| (class 2 or 3) | | | | | | | | |
| Compatible with | ✓ | ✓ | ✓ | ✓ | ✓ | ✓ | ✓ | ✓ |
| manufacturing | | | | | | | | |
| process | | | | | | | | |

BHT-butylated hydroxytoluene
MEK-methyl ethyl ketone
EtAc-ethyl acetate
THF-tetrahydrofuran

The non-limiting exemplary and comparative solvent systems listed in Table 1 were prepared by combining the components of the solvent system at the indicated concentrations. The resulting mixture was then mixed at room temperature until a colorless, clear mixture was obtained. Compound of Formula (I) was then added in the indicated amount and the resulting mixture was mixed at room temperature until a yellow/orange translucent mixture was obtained.

### Example 2: Solubility and Stability Studies of Solvent Systems

The solubility and stability of solvent systems were determined. Results are shown in the Table below.

TABLE 2
Solubility and Stability of Solvent Systems

| | EtOH/Acetic | | EtOH/Water/ | | EtOH/Water/ |
|---|---|---|---|---|---|
| | Acid | EtOH/Water | Acetic Acid | EtOH/Water | Acetic Acid |
| Solvent Criteria | (99.5:0.5) | (98:2) | (99.4:0.5:0.1) | (90:10) | (89.5:10:0.5) |
| Solubility >2% w/w | ✓ | ✓ | ✓ | ✓ | ✓ |
| Storage stability | X | X | X | ✓ | ✓ |

As can be seen, only the ethanol/water (90:10) solvent system and the ethanol, water, acetic acid (89.5:10:0.5) solvent system exhibited both solubility and stability.

### Example 3: Preparation of Coated Balloons

A solvent system was prepared by combining ethanol and water and acetic acid in a volume ratio of 89.5:10:0.5. The resulting mixture was then mixed at room temperature until a colorless, clear mixture was obtained. Compound of Formula (I) was then added in the indicated amount and the resulting mixture was mixed at room temperature until a yellow/orange translucent mixture was obtained. This mixture was applied to a 5 mm×60 mm balloon by a pipetting method. The coated balloon was then dried to evaporate the solvent. A coated balloon with a coating thickness of 10 μg/mm² was obtained. The calculated amount of Compound of Formula (I) on the coated balloon was 7759 μg.

### Example 4: Coated Balloon Sterilization

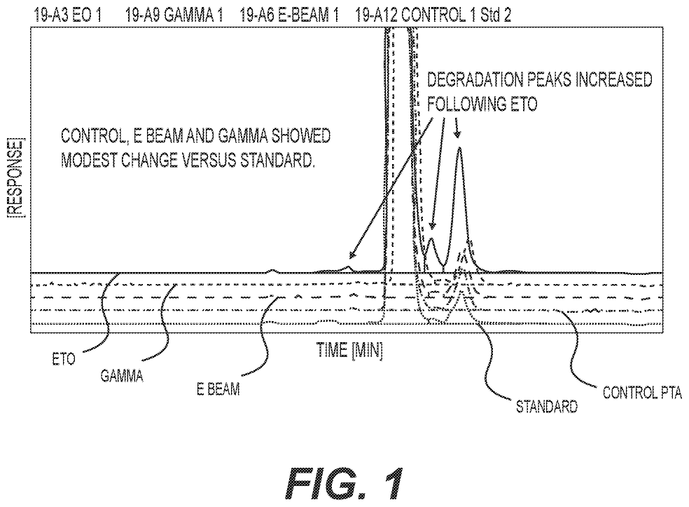

Coated balloons from Example 3 were sterilized either by ethylene oxide sterilization (EO), gamma sterilization (Gamma) or electron beam sterilization (E-Beam). A group of control balloons was left untreated (Control). After sterilization, a sample of the coating on each of the coated balloons was removed and analyzed. The results are summarized in Table 3 and shown in FIG. 1. It was surprisingly found that in a coating comprising the Compound of Formula (I), Compound of Formula (I) is stable after the coated article was subjected to a radiation sterilization process, specifically gamma irradiation and electron beam processing. No change or minimal increase in impurity concentration was observed.

TABLE 3
UPLC analytical results of a Compound of Formula
(I) coated balloon subjected to sterilization.

| Coated Balloon | Compound of Formula (I) | Total impurities |
|---|---|---|
| Sample | (HPLC area-%) | (HPLC area-%) |
| Standard | 98.97 | 1.03 |
| Control 1 | 98.32 | 1.68 |
| Control 2 | 98.33 | 1.67 |
| Control 3 | 98.33 | 1.67 |
| EO 1 | 94.75 | 5.25 |
| EO 2 | 94.26 | 5.74 |
| EO 3 | 94.35 | 5.65 |
| E-Beam 1 | 98.22 | 1.78 |
| E-Beam 2 | 98.33 | 1.67 |
| E-Beam 3 | 98.37 | 1.63 |
| Gamma 1 | 98.32 | 1.68 |
| Gamma 2 | 98.37 | 1.63 |
| Gamma 3 | 98.28 | 1.72 |

### Example 5: Packaging of Coated Balloon

Packaged coated balloons were prepared as follows. A coated balloon from Example 3 was placed in a foil pouch. The concentration of oxygen within the foil pouch was reduced by purging with nitrogen. A desiccant was added to the foil pouch before sealing the oxygen-purged foil pouch comprising the coated balloon and the desiccant.

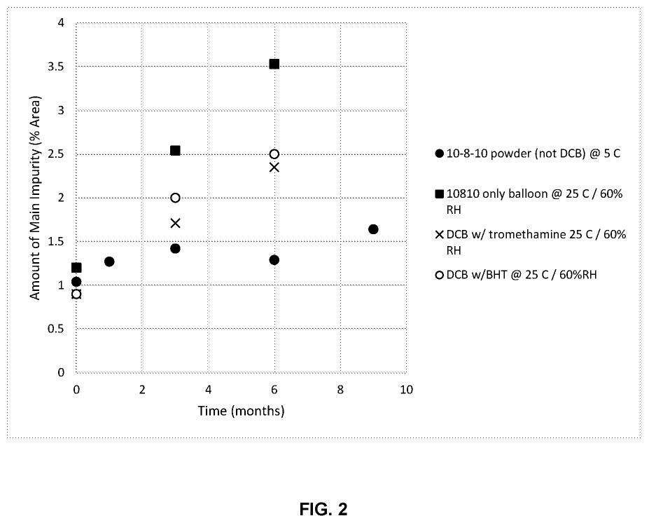

At various time points after packaging, a coated balloon was taken out of its packaging. A sample of the coating on the coated balloon was removed and analyzed. The results are shown in FIG. 2. It was surprisingly found that the increase in impurity concentration was minimal where the coated balloon was stored in a reduced oxygen and reduced moisture packaging.

### Example 6: Room Temperature Stability

Angioplasty balloons coated with the Compound of Formula (I) (“10-8-10 compound”) were tested for their stability at 25° C. over a period of 0, 3, 6, and 12 months. The products described in Table 4 were placed in storage at 25° C.

TABLE 4
Room Temperature Stability Test

| | Coating | Coating |
|---|---|---|
| Article description | Composition | weight |
| 10-8-10 coated balloon, in low | 100% compound | 10 μg/mm² |
| moisture vapor transmission rate | of Formula (I) | |
| (MVTR) primary packaging (Control) | | |
| 10-8-10 + BHT coated balloon, | 99.76% compound | |
| in low MVRT primary packaging | of Formula (I); | |
| | 0.24% BHT | |
| 10-8-10 + tromethamine coated | 99.8% compound | |
| balloon, in low MVRT primary | of Formula (I); | |
| packaging | 0.2% Tromethamine | |

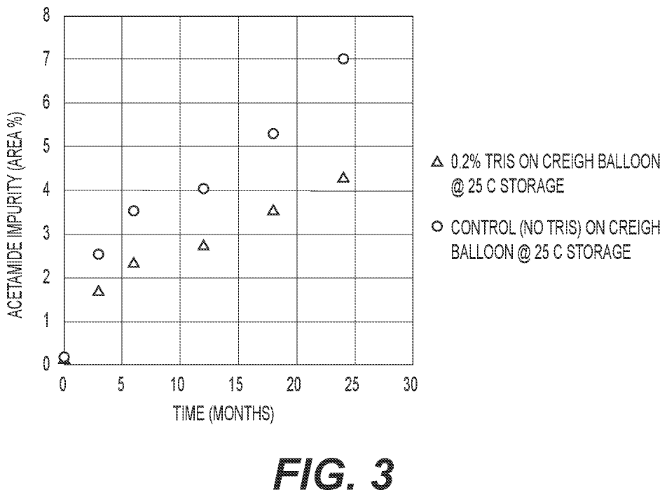

Samples of each article were taken after 0, 3, 6, 12, 18 and 24 months of storage at 25° C., 60% RH. As can be seen in the results in FIG. 3, surprisingly lower amounts of the main impurity were detected by LC in the coatings comprising BHT or tromethamine than the coating comprising only the Compound of Formula (I) at each of the tested times.

What is claimed is:

1. A composition comprising:
at least one active agent chosen from a Compound of Formula (I): 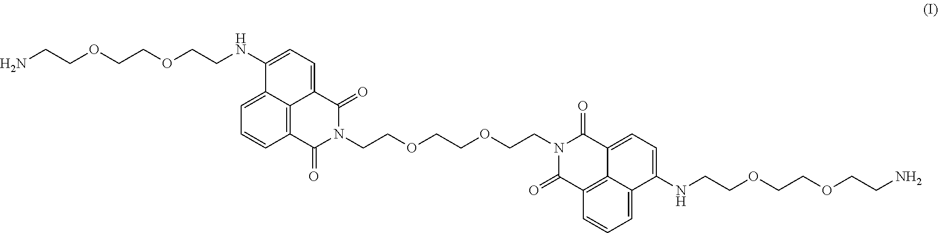 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]
and pharmaceutically acceptable salts thereof; and
a solvent system comprising ethanol, water, and acetic acid,
wherein the at least one active agent does not degrade in the solvent system for at least 8 hours, and
wherein at least 2% w/w of the at least one active agent remains solubilized in the composition for at least 8 hours.

2. A composition according to claim 1, wherein the at least one active agent is 2,2′-((((((((ethane-1,2-diylbis(oxy))bis(ethane-2,1-diyl))bis(1,3-dioxo-2,3-dihydro-1 H-benzo[de]isoquinoline-2,6-diyl))bis(azanediyl))bis(ethane-2,1-diyl))bis(oxy))bis(ethane-2,1 -diyl))bis(oxy))bis(ethan-1-aminium) diacetate.

3. A composition according to claim 1, wherein the at least one active agent is 2,2′-((((((((ethane-1,2-diylbis(oxy))bis(ethane-2,1-diyl))bis(1,3-dioxo-2,3-dihydro-1 H-benzo[de]isoquinoline-2,6-diyl))bis(azanediyl))bis(ethane-2,1-diyl))bis(oxy))bis(ethane-2,1-diyl))bis(oxy))bis(ethan-1-aminium).

4. The composition according to claim 1 or 2, wherein an amount of about 2%, 2.2%, 2.4%, 2.6%, 2.8%, 3%, 3.2%, 3.4%, 3.6%, 3.8%, or 4% w/w of the at least one active agent remain solubilized in the composition for at least 8 hours.

5. The composition according to claim 1 or 2, wherein at least 2% w/w of the at least one active agent remains solubilized in the composition for 10, 12, 14, 16, 18, 20, 22, 24, 30, 36, or 48 hours.

6. The composition according to any one of claims 1-3, wherein an amount of about 2% to about 10% w/w of the at least one active agent remains solubilized in the composition for at least 8 hours.

7. The composition according to any one of claims 1-3, wherein an amount of about 2% to about 10% w/w of the at least one active agent remains solubilized in the composition for 10, 12, 14, 16, 18, 20, 22, 24, 30, 36, or 48 hours.

8. The composition according to claim 1 or 2, wherein an amount of about 5% w/w of the at least one active agent remains solubilized in the composition for at least 8 hours.

9. The composition according to claim 1 or 2, wherein at least 5% w/w of the at least one active agent remains solubilized in the composition for 10, 12, 14, 16, 18, 20, 22, 24, 30, 36, or 48 hours.

10. The composition according to claim 1 or 2, wherein the at least one active agent does not degrade in the solvent system for 10, 12, 14, 16, 18, 20, 22, 24, 30, 36, or 48 hours.

11. The composition according to claim 1, wherein the solvent system comprises ethanol in an amount ranging from 80% to 99.2% v/v.

12. The composition according to claim 1, wherein the solvent system comprises acetic acid in an amount ranging from 0.05% to 5% v/v.

13. The composition according to claim 1, wherein the solvent system comprises water in an amount ranging from 0.6% to 15% v/v.

14. The composition according to claim 1, wherein the solvent system comprises a volume ratio of ethanol to water to acetic acid is 89.5:10:0.5.

15. The composition according to claim 1, wherein the solvent system comprises ethanol and water.

16. The composition according to claim 13, wherein the solvent system comprises ethanol in an amount ranging from 80% to 97% v/v.

17. The composition according to claim 13, wherein the solvent system comprises water in an amount ranging from 3% to 20% v/v.

18. The composition according to claim 13, wherein the solvent system comprises a volume ratio of ethanol to water is 90:10.

19. The composition according to claim 1, further comprising at least one antioxidant.

20. The composition according to claim 17, wherein the at least one antioxidant is chosen from butylated hydroxytoluene (BHT) and tromethamine.

21. The composition according to claim 1, further comprising at least one Lewis acid.

22. The composition according to claim 19, wherein the at least one Lewis acid is Na⁺.

23. The composition according to claim 1, further comprising at least one antioxidant and at least one Lewis acid.

24. The composition according to claim 23, wherein the antioxidant is chosen from butylated hydroxytoluene (BHT) and tromethamine, and the at least one Lewis acid is Na⁺.

25. A coated balloon prepared by coating a balloon with a composition according to claim 1.

26. An angioplasty balloon prepared by coating an angioplasty balloon with a composition according to claim 1.

27. A method of stabilizing a coating composition comprising:
dissolving at least one active agent chosen from a Compound of Formula (I): 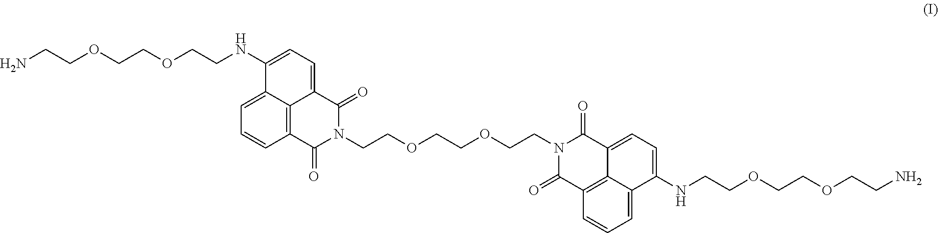 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]
and pharmaceutically acceptable salts thereof in a solvent system comprising ethanol, acetic acid, and water,
wherein the total amount of impurities in the coating composition 1 week after preparation of the coating composition is less than three-fold the total amount of impurities initially present in the composition.

28. The method according to claim 27, wherein the total amount of impurities in the coating composition 1 week after preparation of the coating composition is less than two-fold the total amount of impurities initially present in the composition.

29. The method according to claim 27, wherein the total amount of impurities in the coating composition 1 week after preparation of the coating composition is substantially equal to the total amount of impurities initially present in the composition.

30. The method according to claim 27, wherein the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium) diacetate.

31. The method according to claim 27, wherein the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium).

32. The method according to claim 27, wherein the solvent system comprises ethanol in an amount ranging from 80% to 99.2% v/v.

33. The method according to claim 27, wherein the solvent system comprises acetic acid in an amount ranging from 0.05% to 5% v/v.

34. The method according to claim 27, wherein the solvent system comprises water in an amount ranging from 0.6% to 15% v/v.

35. The method according to claim 27, wherein the solvent system comprises a volume ratio of ethanol to water to acetic acid is 89.5:10:0.5.

36. The method according to claim 27, wherein the solvent system comprises ethanol and water.

37. The method according to claim 27, wherein the solvent system comprises ethanol in an amount ranging from 80% to 97% v/v.

38. The method according to claim 27, wherein the solvent system comprises water in an amount ranging from 3% to 20% v/v.

39. The method according to claim 27, wherein the solvent system comprises a volume ratio of ethanol to water is 90:10

40. The method according to claim 27, wherein the coating composition further comprises at least one antioxidant.

41. The method according to claim 40, wherein the at least one antioxidant is chosen from butylated hydroxytoluene (BHT) and tromethamine

42. The method according to claim 27, wherein the coating composition further comprises at least one Lewis acid.

43. The method according to claim 42, wherein the at least one Lewis acid is Na+.

44. The methods according to claim 27, wherein the coating composition further comprises at least one antioxidant and at least one Lewis acid.

45. The method according to claim 44, wherein the antioxidant is chosen from butylated hydroxytoluene (BHT) and tromethamine, and the at least one Lewis acid is Na⁺.

46. A method of forming a stabilized coating composition comprising:
preparing a coating composition comprising at least one active agent chosen from a Compound of Formula (I): 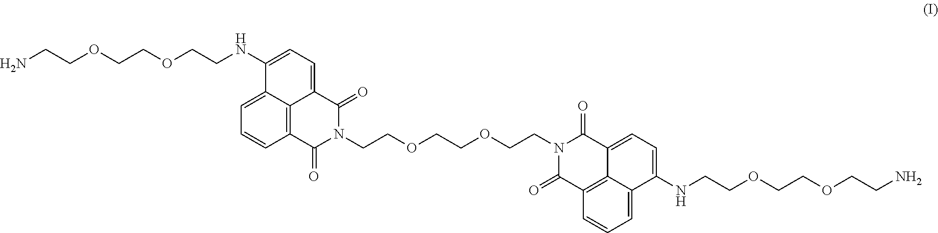 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]
and pharmaceutically acceptable salts thereof and a solvent system comprising ethanol, acetic acid and water,
wherein the total amount of impurities in the coating composition 1 week after preparation of the coating composition is less than three-fold the total amount of impurities initially present in the composition.

47. The method according to claim 46, wherein the total amount of impurities in the coating composition 1 week after preparation of the coating composition is less than two-fold the total amount of impurities initially present in the composition.

48. The method according to claim 46, wherein the total amount of impurities in the coating composition 1 week after preparation of the coating composition is substantially equal to (defined as “less than 1% increase”) the total amount of impurities initially present in the composition.

49. The method according to claim 46, wherein the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium) diacetate.

50. The method according to claim 46, wherein the at least one active agent is 2,2′-((((((((ethane-1,2-diylbis(oxy))bis(ethane-2,1-diyl))bis(1,3-dioxo-2,3-dihydro-1 H-benzo[de]isoquinoline-2,6-diyl))bis(azanediyl))bis(ethane-2,1-diyl))bis(oxy))bis(ethane-2,1 -diyl))bis(oxy))bis(ethan-1-aminium).

51. The method according to claim 46, wherein the solvent system comprises ethanol in an amount ranging from 80% to 99.2% v/v.

52. The method according to claim 46, wherein the solvent system comprises acetic acid in an amount ranging from 0.05% to 5% v/v.

53. The method according to claim 46, wherein the solvent system comprises water in an amount ranging from 0.6% to 15% v/v.

54. The method according to claim 46, wherein the solvent system comprises a volume ratio of ethanol to water to acetic acid is 89.5:10:0.5.

55. The method according to claim 46, wherein the solvent system comprises ethanol and water.

56. The method according to claim 46, wherein the solvent system comprises ethanol in an amount ranging from 80% to 97% v/v.

57. The method according to claim 46, wherein the solvent system comprises water in an amount ranging from 3% to 20% v/v.

58. The method according to claim 46, wherein the solvent system comprises a volume ratio of ethanol to water is 90:10

59. The method according to claim 46, wherein the coating composition further comprises at least one antioxidant.

60. The method according to claim 59, wherein the at least one antioxidant is chosen from butylated hydroxytoluene (BHT) and tromethamine.

61. The method according to claim 46, wherein the coating composition further comprises at least one Lewis acid.

62. The method according to claim 61, wherein the at least one Lewis acid is Na⁺.

63. The method according to claim 59, wherein the coating composition further comprises at least one antioxidant and at least one Lewis acid.

64. The method according to claim 63, wherein the antioxidant is chosen from butylated hydroxytoluene (BHT) and tromethamine, and the at least one Lewis acid is Na⁺.

65. A method of sterilizing a coated article comprising
subjecting the coated article comprising a coating to a radiation sterilization process,
wherein there is less than 25% increase in the total amount of impurities present in the coating after sterilization,
wherein the coating of the coated article comprises at least one active agent chosen from a Compound of Formula (I): 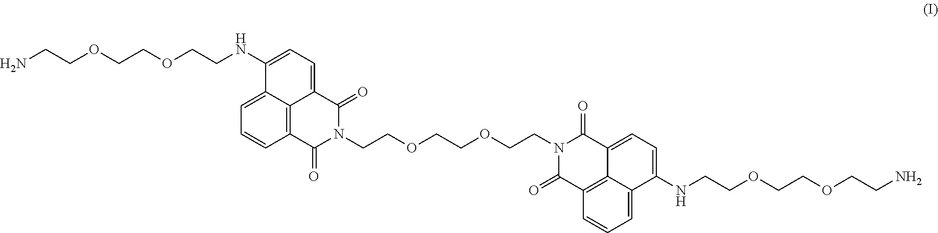 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]
and pharmaceutically acceptable salts thereof.

66. The method according to claim 65, wherein the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium) diacetate.

67. The method according to claim 65, wherein the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium)

68. The method according to claim 65, wherein the radiation sterilization process is chosen from nitrogen dioxide sterilization, gamma irradiation and electron beam processing.

69. The method according to claim 65, wherein there is less than 10% increase in the total amount of impurities present in the coating after sterilization.

70. The method according to claim 65, wherein there is less than 5% increase in the total amount of impurities present in the coating after sterilization.

71. The method according to claim 65, wherein there is less than 1% increase in the total amount of impurities present in the coating after sterilization.

72. The method according to claim 65, wherein the coating of the coated article further comprises at least one antioxidant.

73. The method according to claim 72, wherein the at least one antioxidant is chosen from butylated hydroxytoluene (BHT) and tromethamine.

74. The method according to claim 65, wherein the coating of the coated article further comprises at least one Lewis acid.

75. The method according to claim 74, wherein the at least one Lewis acid is Na⁺.

76. A sterilized coated article produced by the method according to claim 65.

77. A method for preparing an article coated with a stabilized coating comprising at least one active agent chosen from a Compound of Formula (I) and pharmaceutically acceptable salts thereof, the method comprising:
preparing a coating composition comprising at least one active agent chosen from a Compound of Formula (I): 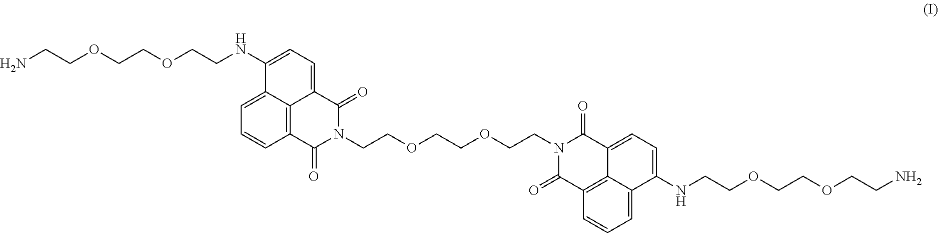 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]
and pharmaceutically acceptable salts thereof and a solvent system comprising ethanol, acetic acid, and water;
applying the coating composition to at least a portion of a surface of an article;
evaporating the solvent system to form a coated article;
packaging said coated article in a sealable container;
reducing the concentration of oxygen from within the container;
adding at least one desiccant to said container; and
sealing said oxygen-purged container comprising the coated article and at least one desiccant.

78. The method according to claim 77, wherein the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium) diacetate.

79. The method according to claim 77, wherein the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium)

80. The method according to claim 77 or 78, wherein the at least one active agent does not degrade more than 10% in 24 months after packaging.

81. The method according to claim 77 or 78, wherein the at least one active agent does not degrade more than 5% in 24 months after packaging.

82. The method according to claim 77 or 78, wherein the at least one active agent does not degrade more than 1% in 24 months after packaging.

83. The method according to claim 77, wherein the concentration of oxygen within the container is reduced by purging with nitrogen.

84. The method according to claim 77, wherein the sealable container is a foil pouch.

85. The method according to claim 77, wherein the desiccant is a silica gel packet.

86. The method according to claim 77, wherein the coating composition and/or the coating further comprises at least one antioxidant.

87. The method according to claim 86, wherein the at least one antioxidant is chosen from butylated hydroxytoluene (BHT) and tromethamine.

88. The method according to claim 77, wherein the coating composition and/or the coating further comprises at least one Lewis acid.

89. The method according to claim 88, wherein the at least one Lewis acid is Na+.

90. The method according to claim 77, wherein the coating composition and/or the coating further comprises at least one antioxidant and at least one Lewis acid.

91. The composition according to claim 90, wherein the antioxidant is chosen from butylated hydroxytoluene (BHT) and tromethamine, and the at least one Lewis acid is Na⁺.

92. A method for stabilizing a coating of an article having a coating comprising at least one active agent chosen from a Compound of Formula (I) and pharmaceutically acceptable salts thereof, the method comprising:
preparing a coating composition comprising at least one active agent chosen from a Compound of Formula (I): 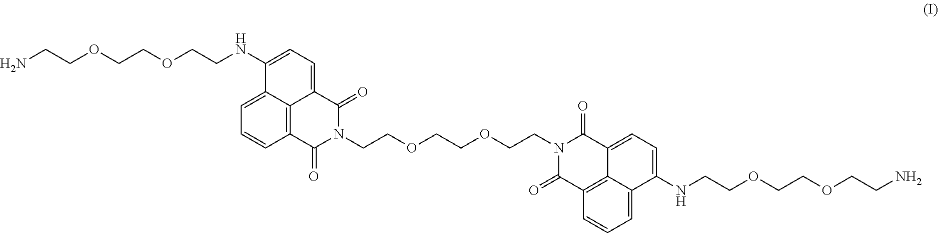 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]
and pharmaceutically acceptable salts thereof and a solvent system comprising ethanol, acetic acid and water;
coating at least a portion of a surface of an article with said coating composition to form a coated article;
packaging said coated article in a sealable container;
reducing the concentration of oxygen from within the container;
adding at least one desiccant to said container; and
sealing said oxygen-purged container comprising the coated article and at least one desiccant.

93. The method according to claim 92, wherein the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium) diacetate.

94. The method according to claim 92, wherein the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium)

95. The method according to claim 92 or 93, wherein the at least one active agent does not degrade more than 10% in 24 months after packaging.

96. The method according to claim 92 or 93, wherein the at least one active agent does not degrade more than 5% in 24 months after packaging.

97. The method according to claim 92 or 93, wherein the at least one active agent does not degrade more than 1% in 24 months after packaging.

98. The method according to claim 92, wherein the concentration of oxygen within the container is reduced by purging with nitrogen.

99. The method according to claim 92, wherein the sealable container is a foil pouch.

100. The method according to claim 92, wherein the desiccant is a silica gel packet.

101. The method according to claim 92, wherein the coating composition and/or the coating further comprises at least one antioxidant.

102. The method according to claim 101, wherein the at least one antioxidant is chosen from butylated hydroxytoluene (BHT) and tromethamine.

103. The method according to claim 92, wherein the coating composition and/or the coating further comprises at least one Lewis acid.

104. The method according to claim 103, wherein the at least one Lewis acid is Na⁺.

105. The method according to claim 92, wherein the coating composition and/or the coating further comprises at least one antioxidant and at least one Lewis acid.

106. The method according to claim 105, wherein the antioxidant is chosen from butylated hydroxytoluene (BHT) and tromethamine, and the at least one Lewis acid is Na⁺.

107. A room temperature storage-stable packaged article coated with a coating comprising at least one active agent chosen from a Compound of Formula (I) and pharmaceutically acceptable salts thereof, comprising:
a sealable container containing, in a reduced oxygen atmosphere, the article coated with the coating comprising at least one active agent chosen from a Compound of Formula (I): 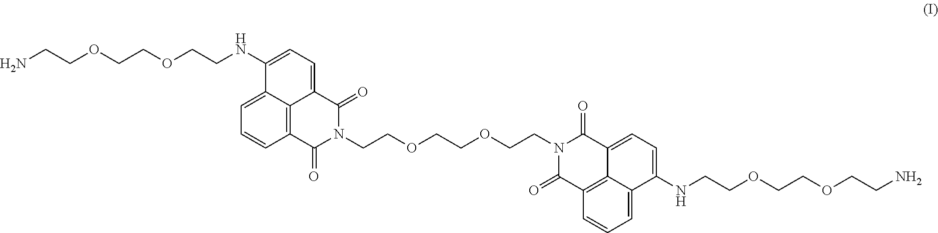 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]
and pharmaceutically acceptable salts thereof; and
at least one desiccant.

108. The method according to claim 107, wherein the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium) diacetate

109. The method according to claim 107, wherein the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium).

110. The method according to claim 107 or 108, wherein the at least one active agent does not degrade more than 10% in 24 months after packaging.

111. The method according to claim 107 or 108, wherein the at least one active agent does not degrade more than 5% in 24 months after packaging.

112. The method according to claim 107 or 108, wherein the at least one active agent does not degrade more than 1% in 24 months after packaging.

113. The method according to claim 107, wherein the desiccant is a silica gel packet.

114. The method according to claim 107, wherein the coating of the coated article further comprises at least one antioxidant.

115. The method according to claim 114, wherein the at least one antioxidant is chosen from butylated hydroxytoluene (BHT) and tromethamine.

116. The method according to claim 107, wherein the coating of the coated article further comprises at least one Lewis acid.

117. The method according to claim 116, wherein the at least one Lewis acid is Na⁺.

118. The method according to claim 107, wherein the coating of the coated article further comprises at least one antioxidant and at least one Lewis acid.

119. The method according to claim 118, wherein the antioxidant is chosen from butylated hydroxytoluene (BHT) and tromethamine, and the at least one Lewis acid is Na⁺.

120. A coating comprising
at least one active agent chosen from a Compound of Formula (I): 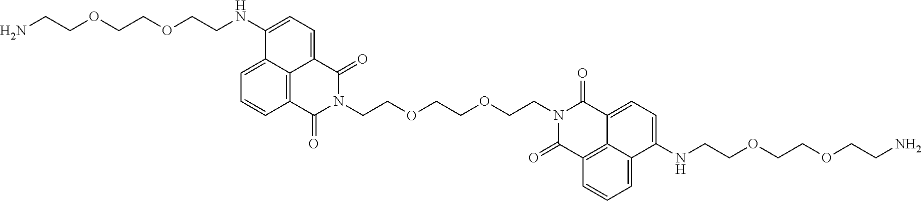 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]
and pharmaceutically acceptable salts thereof,
wherein the coating covers at least a portion of a surface of an article.

121. The coating of claim 120, wherein the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium) diacetate.

122. The coating of claim 120, wherein the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium).

123. The coating of any one of claims 120-122, further comprising acetic acid.

124. The coating of any one of claims 120-123, further comprising at least one antioxidant.

125. The coating of claim 124, wherein the at least one antioxidant is chosen from butylated hydroxytoluene (BHT) and tromethamine.

126. The coating according to any one of claims 120-123, further comprising at least one Lewis acid.

127. The coating according to claim 126, wherein the at least one Lewis acid is Na⁺.

128. The coating according to any one of claims 120-123, further comprising at least one antioxidant and at least one Lewis acid.

129. The coating according to claim 128, wherein the antioxidant is chosen from butylated hydroxytoluene (BHT) and tromethamine, and the at least one Lewis acid is Na⁺.

130. The coating according to any one of claims 120-129, wherein the article is an angioplasty balloon.

131. The coating according to any one of claims 120-130, wherein there is a 0% to less than 30% increase in a total amount of impurities in the coating 24 months after a coating article is packed in comparison the total amount of impurities initially present in the coating.

132. The coating according to any one of claims 120-130, wherein there is a 0% to less than 30% increase in a total amount of impurities in the coating after sterilization.

133. The coating of any one of claims 120-132, wherein the coating is in a dry state.

134. The coating of any one of claims 120-132, wherein the coating is in a liquid state.

135. The coating of any one of claims 120-134, wherein the coating comprises about 90-99.8% by weight % of the at least one active agent.

136. The coating of any one of claims 120-135, wherein the coating has a thickness of about 5 μg/mm² to about 10 μg/mm².

137. The coating of any one of claims 120-136, wherein the coating has a thickness of about 7.5 μg/mm².

138. The coating of any one of claims 120-136, wherein the coating has a thickness of about 6.5 μg/mm².

139. The coating of any one of claims 120-135, wherein the coating has a thickness of about 1 μg/mm² to about 5 μg/mm².

140. The coating of any one of claims 120-135 and 139, wherein the coating has a thickness of about 2.3 μg/mm².
